(19) United States
(12) Patent Application Publication
(10) Pub. No.: US 2024/0110140 A1
(43) Pub. Date: Apr. 4, 2024

(54) Cover For Cell Culture Apparatus

(71) Applicant: NOVOCURE GMBH, Root D4 (CH)

(72) Inventor: Elie Yaacobi, Haifa (IL)

(21) Appl. No.: 18/476,536

(22) Filed: Sep. 28, 2023

Related U.S. Application Data
(60) Provisional application No. 63/411,774, filed on Sep. 30, 2022.

(51) Int. Cl. C12M 1/00; C12M 1/22

(52) U.S. Cl. CPC C12M 23/38; C12M 23/10

(57) ABSTRACT

A cover having a central axis and a circumference. The cover overlies an interior space defined by a container. The cover includes a base. The base has a center point that is intersected by the central axis of the cover. The base has an outer portion that extends radially inwardly from the circumference toward the central axis. The outer portion defines a bottom surface of the cover. The base has an inner portion positioned radially inwardly of the outer portion. The inner portion of the base has an apex that is upwardly spaced from the outer portion along the central axis. The inner portion of the base directs condensate radially outwardly from the apex toward the outer portion of the base. Optionally, the cover has a peripheral wall that extends from the outer portion of the base.

### CROSS-REFERENCE TO RELATED APPLICATION

This application claims priority to and the benefit of the filing date of U.S. Provisional Patent Application No. 63/411,774, filed Sep. 30, 2022, the entirety of which is hereby incorporated by reference herein.

### BACKGROUND

Tumor Treating Fields (TTFields) therapy is a proven approach for treating tumors using alternating electric fields at frequencies between 50 kHz-1 MHz, more commonly, 100-500 kHz. In current commercial systems, the alternating electric fields are induced by electrode assemblies (e.g., arrays of capacitively coupled electrodes, also called transducer arrays) placed on opposite sides of a subject's body. When an AC voltage is applied between opposing electrode assemblies, an AC current is coupled through the electrode assemblies and into the subject's body. Higher currents are strongly correlated with higher efficacy of treatment.

The INOVITRO system supplied by NOVOCURE is an existing system for studying TTFields in vitro. The INOVITRO system includes ceramic culture dishes constructed to capacitively couple electric field into the cell culture while the cell cultures are maintained at a controlled temperature. This is used to simulate the capacitive coupling of the electric fields into a patient's body. However, due to the heat associated with the electric field generation, condensation within the cell culture structure is possible.

### SUMMARY

Disclosed herein, in one aspect, is a cover having a central axis and a circumference and being configured to overlie an interior space defined by a container. The cover can include a base. The base can have a center point that is intersected by the central axis of the cover. The base can have an outer portion that extends radially inwardly from the circumference toward the central axis. The outer portion can define a bottom surface of the cover. The base can also have an inner portion positioned radially inwardly of the outer portion. The inner portion of the base can have an apex that is upwardly spaced from the outer portion along the central axis. The inner portion of the base can be configured to direct condensate radially outwardly from the apex toward the outer portion of the base. Optionally, the cover can include a peripheral wall that extends from the base and defines the circumference of the cover. The peripheral wall can have a top edge and a bottom edge.

An apparatus can include a dish having a peripheral wall that defines a top surface of the dish. The apparatus can further include a cover as disclosed herein, with the bottom surface of the cover resting on the top surface of the dish. In use, cellular media can be positioned within an insert that is received within the peripheral wall of the dish, and an electric field (e.g., a TTField) can be applied to the cellular media.

### BRIEF DESCRIPTION OF THE DRAWINGS

FIG. 1 is a perspective view of an apparatus as disclosed herein.

FIG. 2 is a partial section view of the apparatus of FIG. 1, taken at section line 2-2 of FIG. 1.

FIG. 3A is a bottom plan view of a cover as disclosed herein. FIG. 3B is a section view of the cover of FIG. 3A, taken at section line 3B-3B of FIG. 3A. FIG. 3C is a section view of a cover having no peripheral wall. FIG. 3D is a section view of a cover having a peripheral wall extending downwardly from the base of the cover.

FIG. 4 is an isolated perspective view of a peripheral wall of a dish having associated electrodes as disclosed herein.

FIG. 5 is a top view of the peripheral wall of the dish of FIG. 4.

Various embodiments are described in detail below with reference to the accompanying drawings, wherein like reference numerals represent like elements, and wherein descriptions of like elements may not be repeated for every embodiment, but may be considered to be the same if previously described herein.

### DETAILED DESCRIPTION

This application describes exemplary cover/lid/cap structures that may be used, e.g., for covering or overlying containers that contain cell culture media that are subjected to TTFields in vitro.

The present invention can be understood more readily by reference to the following detailed description, examples, drawings, and claims, and their previous and following description. However, it is to be understood that this invention is not limited to the specific apparatuses, devices, systems, and/or methods disclosed unless otherwise specified, and as such, of course, can vary.

Headings are provided for convenience only and are not to be construed to limit the invention in any manner. Embodiments illustrated under any heading or in any portion of the disclosure may be combined with embodiments illustrated under the same or any other heading or other portion of the disclosure.

Any combination of the elements described herein in all possible variations thereof is encompassed by the invention unless otherwise indicated herein or otherwise clearly contradicted by context.

As used in the specification and the appended claims, the singular forms “a,” “an” and “the” can include plural referents unless the context clearly dictates otherwise. Thus, for example, unless the context clearly dictates otherwise, disclosure of “a holder” can represent disclosure of embodiments in which only a single such holder is provided, as well as embodiments in which a plurality of such holders are provided.

### Introduction

Existing systems, such as the INOVITRO system supplied by NOVOCURE, can be used to study application of alternating electric fields (e.g., TTFields) in vitro. The INOVITRO system includes ceramic culture dishes constructed to capacitively couple electric field into the cell culture while the cell cultures are maintained at a controlled temperature, thereby simulating the capacitive coupling of the electric fields into a patient's body. However, the heat generated within such systems can lead to condensation within the enclosed area housing the cell culture. With existing systems, there is a risk that such condensation can accumulate on the cover of the housing and then fall into the cell culture, thereby having a negative impact on the performance and usefulness of the system.

### Apparatus for Culturing Cells While Applying Alternating Electric Fields (e.g., TTFields)

In some exemplary aspects, systems for studying alternating electric fields (e.g., TTFields) in vitro can include a dish-like apparatus for applying electric fields to a sample and for observing the sample using an inverted microscope while the sample is illuminated by a light source. This embodiment can facilitate time lapse microscopy during the application of the alternating electric field (e.g., TTFields). The inverted microscope that is used to observe the sample can have a stage and an objective. The dish-like apparatus may be designed to sit directly on the stage of the inverted microscope while the inverted microscope is positioned in an incubator (which optionally may provide any necessary gases to the sample). In some aspects, in order to allow light to flow through the sample for the purpose of microscopy experiments or other imaging techniques, the dish-like apparatus can have a bottom panel (not shown) with a transparent region. Optionally, the floor of a Petri dish can serve as the bottom panel. Preferably, the transparent region does not introduce optical distortions. In those embodiments that use the floor of a Petri dish to serve as the bottom panel, Ibidi® dishes may be used to minimize optical distortions.

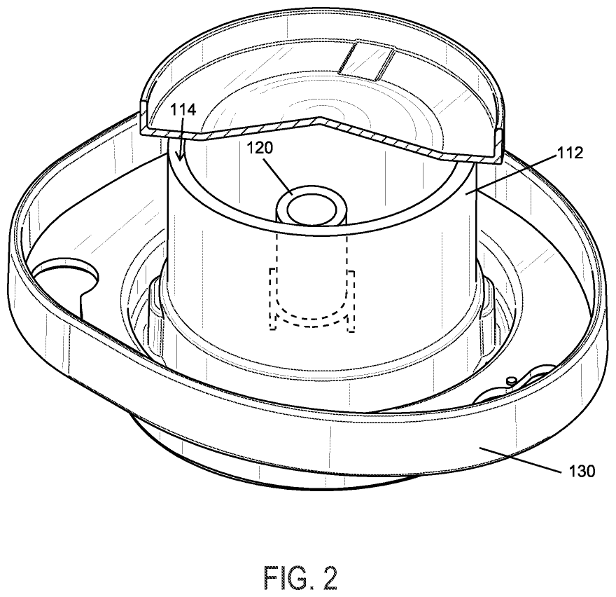

In some aspects, peripheral walls 112 (e.g., ceramic sidewalls) (see FIG. 2) are affixed to the bottom panel such that the peripheral walls 112 (e.g., ceramic sidewalls) and the bottom panel cooperate to form a container 110 that is shaped and dimensioned for holding the sample. In embodiments where only a portion of the bottom panel is transparent, the peripheral walls (e.g., ceramic sidewalls) can be affixed to the bottom panel at a position that surrounds the transparent region. In embodiments where the entire bottom panel is transparent, the peripheral walls (e.g., ceramic sidewalls) 112 may be affixed to the bottom panel anywhere (e.g. close to the perimeter of the bottom panel). In this case, a portion of the bottom panel that lies within the boundaries of the peripheral walls (e.g., ceramic sidewalls) 112 will serve as the transparent region. The peripheral walls (e.g., ceramic sidewalls) 112 are preferably made of a high capacitance material (e.g. PMN-PT).

The bottom panel, the transparent region of the bottom panel, and the peripheral walls (e.g., ceramic sidewalls) 112 can be sized and shaped to facilitate positioning of the container on the stage of the inverted microscope so that when the sample is positioned in the container, light emanating from the light source is free to travel along an optical path that passes through the sample, through the transparent region of the bottom panel 20, and into the objective of the inverted microscope.

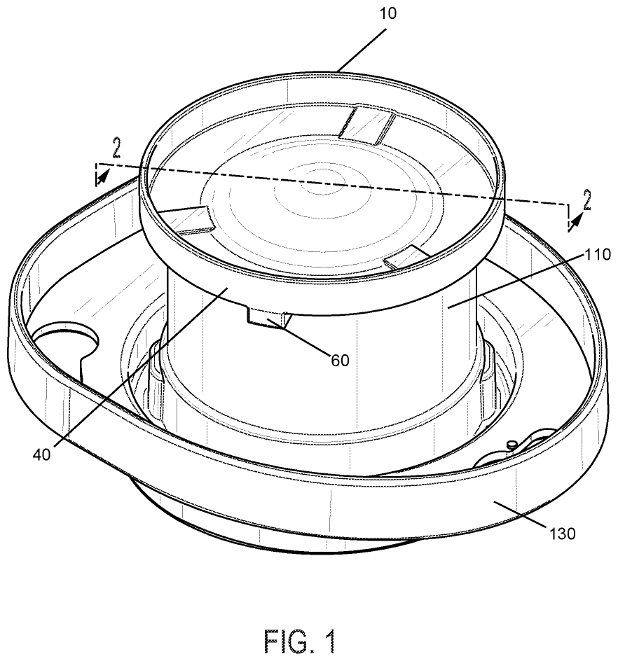

In some embodiments, and as shown in FIGS. 1-2, the peripheral walls (e.g., ceramic sidewalls) 112 can be formed from a single cylindrical tube. In these embodiments, the peripheral walls (e.g., ceramic sidewalls) 112 can have a single cylindrical outer surface. In alternative embodiments, peripheral walls (e.g., ceramic sidewalls) 112 with different shapes may be used (e.g. square or octagonal). In these embodiments, the peripheral walls (e.g., ceramic sidewalls) 112 can have two or more outer surfaces.

In some embodiments, the peripheral walls (e.g., ceramic sidewalls) 112 can be mounted to the bottom panel using an adhesive (e.g., biocompatible glue or cement). In alternative embodiments, the peripheral walls (e.g., ceramic sidewalls) 112 can be mounted to the bottom panel using a screw mount configured to squeeze the peripheral walls (e.g., ceramic sidewalls) and the bottom panel together. For example, an upper housing of the container 110 can connect to a Petri dish using a threaded screw-mount connection which includes a set of external threads (not shown) on the upper housing and a corresponding set of internal threads (not shown) on the Petri dish. In these embodiments, an O-ring can be positioned between the peripheral walls (e.g., ceramic sidewalls) 112 and the bottom panel of the Petri dish such that the O-ring is compressed when the upper housing is screwed into the Petri dish. The O-ring can seal liquids into the volume defined by the bottom panel and the cylindrical sidewalls 112. The upper housing of the container 110 can have an opening through which samples can be inserted into the Petri dish. In exemplary aspects, the upper housing can define the peripheral walls 112.

In alternative embodiments, instead of using a screw mount, an O-ring that has (a) an outer diameter that matches the inner diameter of the Petri dish and (b) an inner diameter that matches the outer diameter of the cylindrical sidewalls 112 can be used, and the cylindrical sidewalls can be jammed into the O-ring to provide an interference fit.

Optionally, when the floor of a glass Petri dish serves as the bottom panel, the vertical walls of the Petri dish can be disposed radially beyond the peripheral walls (e.g., ceramic sidewalls) 112.

It is contemplated that the height of the peripheral walls (e.g., ceramic sidewalls) 112 may be varied to allow different amounts of media to be placed within each container as well as to accommodate possible inserts (e.g. Boyden inserts) 120 (see FIG. 2). In alternative embodiments, tall containers may be obtained by grafting a second cylinder made of a biocompatible material (e.g., glass or polycarbonate) to the top of a short ceramic cylinder. Positioning the ceramic cylinder at the bottom of the container can facilitate the application of alternating electric fields, (e.g., TTFields) to bottom of the container where cells are plated.

Optionally, tubing (a single piece or a plurality of pieces) can be provided to allow for media replacement without the need for removing the dish from the stage of the microscope (not shown) while maintaining sterile conditions.

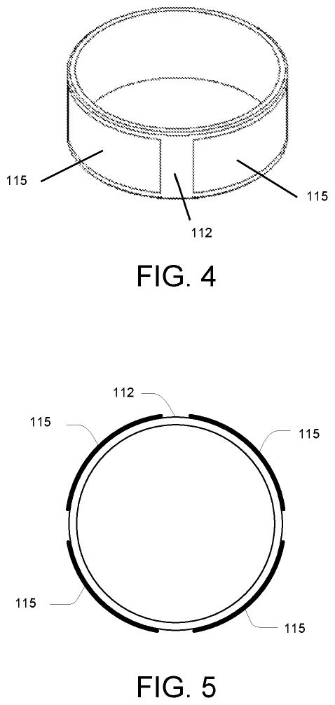

As shown in FIGS. 4-5, a plurality of electrodes 115 can be disposed on the at least one outer surface of the peripheral walls (e.g., ceramic sidewalls) 112 at positions selected so that when the sample is positioned in the container, application of a voltage between the plurality of electrodes 115 induces an electric field through the sample. In some embodiments, at least four electrodes 115 are disposed on the outer surface (or surfaces) of the peripheral walls (e.g., ceramic sidewalls) 112 at positions selected so that when the sample is positioned in the container, (a) application of a voltage between a first subset of the at least four electrodes induces an electric field in a first direction through the sample, and (b) application of a voltage between a second subset of the at least four electrodes induces an electric field in a second direction through the sample. The electrodes 115 may be formed on the outer surface of the peripheral walls (e.g., ceramic sidewalls) 112 by painting panels of a conductive material directly onto the outer surface, by applying a thin sheet of a conductive material using a suitable conductive adhesive, by compressing a thin leaf of metal (e.g., gold) directly onto the outer surface of the peripheral walls (e.g., ceramic sidewalls) 112, or by a variety of alternative approaches that will be apparent to persons skilled in the relevant arts.

The electrodes 115 and the region of the peripheral walls (e.g., ceramic sidewalls) 112 beneath the electrodes form capacitive electrodes through which the electric field is coupled into the sample (i.e. the cell culture). An advantage of using a ceramic with a high relative permittivity is that the impedance of the electrodes can be kept low whilst maintaining the walls at a thickness that ensures the mechanical rigidity of the dish-like apparatus.

In exemplary embodiments, and as shown in FIG. 5, the sidewalls 112 can be cylindrical, and four electrodes 115 can be positioned on respective quadrants of the cylindrical sidewalls. A first electrode is disposed at a first position on the cylindrical sidewalls 112 and a second electrode is disposed at a second position on the cylindrical sidewalls that is opposite to the first position. A third electrode is disposed at a third position on the cylindrical sidewalls 112 and a fourth electrode is disposed at a fourth position on the cylindrical sidewalls that is opposite to the third position.

Application of an AC voltage between a first subset of electrodes consisting of the first and second electrodes can induce an electric field in a first direction through the sample. Application of an AC voltage between a second subset of electrodes consisting of the third and fourth electrodes can induce an electric field in a second direction through the sample. When the electrodes are arranged as depicted in FIGS. 4-5, the second direction is perpendicular to the first direction. If one subset of electrodes (e.g. electrodes 41 and 42) is shifted by a small angle (e.g. less than 10°), the second direction will be roughly perpendicular (e.g., within 10 degrees of perpendicular) to the first direction.

A plurality of electrical conductors can be provided, with each of the plurality of electrical conductors providing electrical contact with a respective one of the plurality of electrodes 115 and routing electricity between a given one of those electrodes and a respective corresponding electrical terminal. In embodiments that have at least four electrodes, at least four electrical conductors can be provided, and each of the at least four electrical conductors can provide electrical contact between a respective one of at least four electrical terminals and a respective one of the at least four electrodes. The conductors can optionally be implemented using individual wires, ribbon cables, flex circuits, etc. Each of the conductors can be connected to a respective electrode 115 using any appropriate approach including but not limited to soldering, electrical connectors, etc. In some embodiments, each of the electrical terminals is disposed in a single electrical connector.

Each of the plurality of electrodes 115 and each of the plurality of electrical conductors can be positioned with respect to the transparent region of the bottom panel so as not to interfere with the optical path described above.

The conductors can be used for applying electric fields to a sample that is positioned in the container. For example, an AC voltage between 50 kHz and 10 MHz (e.g., between 50 kHz and 1 MHz or between 50 and 500 kHz) may be applied across the conductors that are wired to a first electrode pair (e.g., the first and second electrodes) and then across the conductors that are wired to a second electrode pair (e.g., the third and fourth electrodes) in an alternating and repeating sequence, thereby causing electric fields with different directions to be generated in the samples that are located in the container in a corresponding alternating and repeating sequence. In alternative embodiments, the voltages may be applied across different combinations of the electrodes 115 in a different sequence to provide alternative field shapes or directions. In alternative embodiments, the voltages may be applied across a single combination of the electrodes 115 in a single direction.

The dish-like apparatus described herein can be useful for various assays such as: watching the evolution of cellular structures in response to an alternating electric field (e.g., TTFields); using fluorescent dyes, GFP-tagged proteins, or other labeled proteins; scanning frequencies to determine the most effective frequency; measuring cells' sensitivity assays to different alternating electric fields (e.g., TTFields) intensities; measuring the diameter of cells; measuring migration rates and directions during alternating electric fields (e.g., TTFields) application; determining alternating electric fields' (e.g., TTFields') effect on cell invasion using a Boyden chamber inserted into the container; determining alternating electric fields' (TTFields') effect intracellular on different structures/molecules within the cell; and determining alternating electric fields' (TTFields') effect on cell grown in 3D structure (e.g. microspheres) using specific inserts which maintain and support the 3D structures (e.g. agarose mesh).

### The Cover

In exemplary aspects, a cover 10 can be configured for positioning over a container 110 to provide an apparatus 100. Optionally, the container 110 can be a dish or dish-like container as further disclosed herein. In exemplary aspects, the cover 10 can be transparent or translucent to permit visibility of the interior of the container 110. In use, the cover 10 can be configured to maintain sterile conditions within the container while minimizing evaporation and allowing for gas exchange. The cover 10 is preferably made of a transparent material that allows light from the microscope condenser to reach the sample (i.e., the cell culture), thereby enabling imaging of the sample using the light from the condenser.

When the cover 10 is positioned over a container 110 as disclosed herein and alternating electric fields (e.g., TTFields) are applied to a cell culture (or other sample) within container, Ohmic losses in the cell culture heat the cell culture medium. This heat can produce condensation within the container, particularly on a bottom surface of the cover (facing the cell culture). As further disclosed herein, the cover 10 is provided with a structure that directs condensate away from the portion of the cover overlying the cell culture medium, thereby avoiding contamination or disruption of the cell culture medium associated with condensate dropping into the cell culture medium. For example, a cover with a flat surface overlying the cell culture medium may allow for accumulation of condensate over the cell culture medium, and condensate will gradually fall into the cell culture medium. In contrast, by providing a cover having sloped surfaces as further disclosed herein, the condensate is not allowed to accumulate over the cell culture medium—instead, it is directed to the outer portion of the cover that does not overlie the cell culture medium. Consequently, the cover helps prevent condensate from accumulating over and falling into the cell culture medium.

Referring to FIGS. 1-3B, the cover 10 can have a central axis 12 and a circumference 40. As used herein, the term “circumference” does not exclude non-circular perimeter shapes. Rather, the term “circumference” should be interpreted to include any outer perimeter shape, including, for example, a circular perimeter, an oval perimeter, a square perimeter, a triangular perimeter, a rectangular perimeter, and the like. However, it should be understood that when the cover is used with a cylindrical container as further disclosed herein, it can be advantageous to use a cover having a round or circular perimeter (circumference). The cover 10 can be configured to overlie an interior space defined by a container 110. The cover 10 can comprise a base 20 having a center point 22 that is intersected by the central axis 12 of the cover.

In various aspects, the base 20 can comprise an outer portion 24 extending radially inwardly from the circumference 40 toward the central axis 12. In exemplary aspects, the outer portion 24 can define a bottom surface 50 of the cover 10. In further aspects, the base 20 can comprise an inner portion 26 positioned radially inwardly of the outer portion 24. In some aspects, the inner portion 26 of the base 20 can extend radially inwardly from and be circumferentially surrounded by the outer portion 24 of the base.

In exemplary aspects, the inner portion 26 of the base 20 comprises an apex 28 that is upwardly spaced from the outer portion 24 along the central axis 12. As used herein, the apex 28 can be formed by a particular region of the inner portion 26, which can be structured as a point or a rounded area. Optionally, the apex 28 of the base 20 can be intersected by the central axis 12 of the cover 10. In some exemplary aspects, the inner portion 26 of the base 20 can have a conical shape or substantially conical shape, such as, for example, a frustoconical shape.

In exemplary aspects, and as further described below, the inner portion 26 of the base 20 can be configured to direct condensate in a direction moving radially outwardly from the apex 28 toward the outer portion 24 of the base.

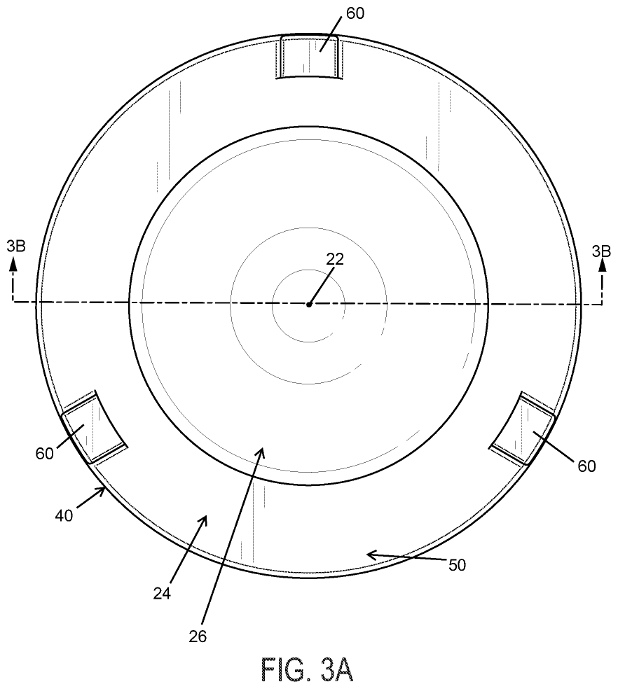

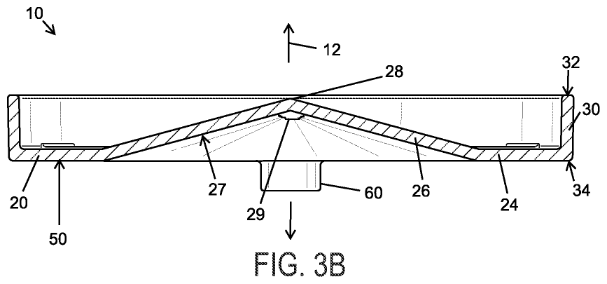

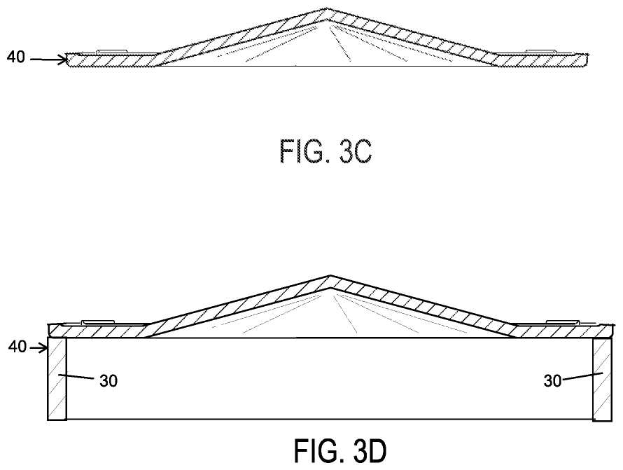

In exemplary aspects, and with reference to FIGS. 2 and 3B, the inner portion 26 of the base 20 can have a sloped surface 27 that extends upwardly from the outer portion 24 of the base. Thus, when moving radially outwardly from the apex 28, the sloped surface 27 extends downwardly, thereby allowing the force of gravity to assist with the guidance of condensate to the outer portion of the cover. Optionally, in some aspects, the sloped surface 27 can be a continuous surface that extends circumferentially about the apex 28. In other aspects, it is contemplated that the inner portion 26 of the base can comprise a plurality of discrete sloped surfaces 27.

In exemplary aspects, within respective vertical planes that intersect the apex 28, opposing sloped surfaces 27 of the inner portion 26 of the base 20 can form an obtuse angle 29 as shown in FIG. 3B. For example, it is contemplated that the obtuse angle 29 can range from about 125 degrees to about 175 degrees or from about 145 degrees to about 165 degrees. In further aspects, it is contemplated that the obtuse angle 29 can be about 150 degrees. Although the sloped surfaces are shown as straight surfaces, it is contemplated that the sloped surfaces can comprise curved portions without departing from the scope of the disclosure. More generally, it is contemplated that the sloped surface(s) 27 can have any shape that is configured to direct fluid (e.g., condensate) from the area of the apex in a downward and radially outward direction toward the outer portion of the base.

Optionally, the cover 10 can further comprise a peripheral wall 30 extending from the base 20 and defining the circumference 40 of the cover. The peripheral wall 30 can have a top edge 32 and a bottom edge 34.

In some aspects, and as shown in FIGS. 1-2 and 3B, the peripheral wall 30 can extend upwardly from the outer portion 24 of the base 20. In these aspects, the peripheral wall 30 of the cover can have a height corresponding to a distance, along the central axis 12, between the top and bottom edges 32, 34 of the peripheral wall. It is contemplated that a distance along the central axis 12, between the bottom edge 32 of the peripheral wall 30 and the apex 28 of the inner portion of the base can be less than, greater than, or equal or substantially equal to the height of the peripheral wall.

In other aspects, and as shown in FIG. 3C, it is contemplated that the peripheral wall 30 can be omitted.

Optionally, in exemplary aspects, the cover 10 can further comprise at least one projection 60 that extends downwardly from the outer portion 24 of the base 20. Optionally, the at least one projection 60 can comprise a plurality of projections. In one example, and as shown in FIG. 3A, the plurality of projections 60 can be equally circumferentially spaced about the outer portion 24 of the base 20. Optionally, it is contemplated that the plurality of projections 60 can be equally radially spaced from the central axis 12. In use, it is contemplated that the plurality of projections 60 can be configured to be positioned radially outwardly of the peripheral wall 112 of the container 110 as further disclosed herein, thereby promoting alignment of the cover with the container and providing stability to the cover 10 during use.

In further aspects, the peripheral wall 30 can extend downwardly from the outer portion 24 of the base 20 of the cover. In these aspects, it is contemplated that the peripheral wall 30 can be configured to be positioned radially outwardly of the peripheral wall 112 of the container 110 as further disclosed herein, thereby promoting alignment of the cover with the container and providing stability to the cover 10 during use. It is further contemplated that projections can be omitted from such embodiments.

In exemplary aspects, and as further disclosed herein, an apparatus 100 can comprise a container 110 (e.g., a dish) having a peripheral wall 112 that defines a top surface 114 of the container. In further exemplary aspects, the bottom surface 50 of the cover 100 rests on the top surface 114 of the container 110 (e.g., dish). When the cover 100 comprises one or more projections 60, it is contemplated that the projections 60 can be positioned radially outwardly of the peripheral wall 112 of the container. For example, the cover 100 can comprise at least one projection 60 that extends downwardly from the outer portion 24 of the base 20 of the cover, and each projection can have an inwardly facing surface that is positioned radially outwardly of the peripheral wall of the dish. In these aspects, it is contemplated that a first area of the outer portion 24 of the base 20 (that defines the bottom surface 50 of the cover) can rest on the top surface 114 of the container, while a second (more outward) area of the outer portion 24 of the base 20 can extend radially outwardly beyond the peripheral wall 112 of the container. Similarly, when the cover 100 comprises a downwardly extending peripheral wall 30, the peripheral wall of the cover can be positioned radially outwardly of the peripheral wall 112 of the container.

In exemplary aspects, the apparatus 100 can further comprise an insert 120 positioned within the container 110 (e.g., dish). In these aspects, the insert 120 can be configured to receive cellular media, and the apex 28 of the cover 10 can overlie the insert. In further aspects, the outer portion 24 of the base 20 of the cover 10 does not overlie the insert. Thus, in use, when condensate is directed from the inner portion 26 of the base to the outer portion 24 of the base, the condensate can fall from the outer portion 24 of the base without entering the insert 120 and contacting the cellular media within the insert.

In still further aspects, the apparatus 100 can comprise a holder 130 that supports the container 110 (e.g., dish) in a vertical position. Optionally, the holder 130 can be configured for mounting or coupling to a microscope stage. In further aspects, the holder 130 can comprise a platform that is configured for engagement with the container 110 (e.g., dish) to support the container in a centered position within the holder.

In use, cellular media can be positioned within the container 110 of the apparatus 100. For example, cellular media can be positioned within an insert 120 that is positioned within the container 110. With the cellular media positioned within the container 110, a method can further comprise applying an electric field to the cellular media. For example, electrodes 115 can be used to generate and apply an alternating electric field (e.g., TTField) to the cellular media within the container. In exemplary aspects, the method can further comprise directing condensate radially outwardly from the inner portion of the cover to the outer portion of the cover. More particularly, as condensate develops or accumulates on the surface of the inner portion of the cover (for example, in the area of the apex), the sloped surfaces of the base can direct the condensate to the outer portion of the cover, thereby ensuring that when the condensate falls, it will not fall into the cellular media within the container or insert.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A cover having a central axis and a circumference, the cover being configured to overlie an interior space defined by a container, the cover comprising:
a base having a center point that is intersected by the central axis of the cover, wherein the base comprises:
   an outer portion extending radially inwardly from the circumference toward the central axis, wherein the outer portion defines a bottom surface of the cover; and
   an inner portion positioned radially inwardly of the outer portion, wherein the inner portion comprises an apex that is upwardly spaced from the outer portion along the central axis,
wherein the inner portion of the base is configured to direct condensate radially outwardly from the apex toward the outer portion of the base.

Aspect 2: The cover of aspect 1, wherein the inner portion of the base extends radially inwardly from and is circumferentially surrounded by the outer portion of the base.

Aspect 3: The cover of aspect 1 or aspect 2, and wherein the apex of the base is intersected by the central axis of the cover.

Aspect 4: The cover of aspect 3, wherein the inner portion of the base has a conical shape.

Aspect 5: The cover of aspect 3 or aspect 4, wherein the inner portion of the base has one or more sloped surfaces that extend upwardly from the outer portion of the base.

Aspect 6: The cover of aspect 5, wherein within respective vertical planes that intersect the apex, opposing sloped surfaces of the inner portion of the base form an obtuse angle.

Aspect 7: The cover of aspect 6, wherein the obtuse angle ranges from about 125 degrees to about 175 degrees.

Aspect 8: The cover of aspect 6, wherein the obtuse angle ranges from about 145 degrees to about 165 degrees.

Aspect 9: The cover of any one of the preceding aspects, further comprising a peripheral wall extending from the outer portion of the base and defining the circumference of the cover.

Aspect 10: The cover of aspect 9, wherein the peripheral wall extends upwardly from the outer portion of the base.

Aspect 11: The cover of aspect 10, wherein the peripheral wall has a top edge and a bottom edge, wherein the peripheral wall has a height corresponding to a distance, along the central axis, between the top and bottom edges of the peripheral wall, and wherein a distance along the central axis, between the bottom edge of the peripheral wall and the apex of the inner portion of the base is less than the height of the peripheral wall.

Aspect 12: The cover of aspect 10, wherein the peripheral wall has a top edge and a bottom edge, wherein the peripheral wall has a height corresponding to a distance, along the central axis, between the top and bottom edges of the peripheral wall, and wherein a distance along the central axis, between the bottom edge of the peripheral wall and the apex of the inner portion of the base is greater than the height of the peripheral wall.

Aspect 13: The cover of aspect 10, wherein the peripheral wall has a top edge and a bottom edge, wherein the peripheral wall has a height corresponding to a distance, along the central axis, between the top and bottom edges of the peripheral wall, and wherein a distance along the central axis, between the bottom edge of the peripheral wall and the apex of the inner portion of the base is substantially equal to the height of the peripheral wall.

Aspect 14: The cover of any one of the preceding aspects, further comprising at least one projection that extends downwardly from the outer portion of the base.

Aspect 15: The cover of aspect 14, wherein the at least one projection comprises a plurality of projections.

Aspect 16: The cover of aspect 15, wherein the plurality of projections are equally circumferentially spaced about the outer portion of the base.

Aspect 17: The cover of aspect 16, wherein the plurality of projections are equally radially spaced from the central axis.

Aspect 18: The cover of aspect 9, wherein the peripheral wall extends downwardly from the outer portion of the base.

Aspect 19: The cover of any one of aspects 1-8, wherein the cover does not comprise a peripheral wall that extends upwardly or downwardly from the outer portion of the base, and wherein the cover comprises at least one projection that extends downwardly from the outer portion of the base.

Aspect 20: An apparatus comprising:
a dish having a peripheral wall that defines a top surface of the dish; and
the cover according to any one of the preceding aspects, wherein the bottom surface of the cover rests on the top surface of the dish.

Aspect 21: The apparatus of aspect 20, further comprising an insert positioned within the dish, the insert being configured to receive cellular media, wherein the apex of the cover overlies the insert.

Aspect 22: The apparatus of aspect 21, wherein the outer portion of the base of the cover does not overlie the insert.

Aspect 23: The apparatus of any one of aspects 20-22, wherein the cover comprises at least one projection that extends downwardly from the outer portion of the base of the cover, and wherein each projection of the at least one projection has an inwardly facing surface that is positioned radially outwardly of the peripheral wall of the dish.

Aspect 24: The apparatus of any one of aspects 20-22, wherein the cover comprises a peripheral wall that extends downwardly from the outer portion of the base of the cover, and wherein the peripheral wall of the cover is positioned radially outwardly of the peripheral wall of the dish.

Aspect 25: The apparatus of any one of aspects 20-24, further comprising a holder that supports the dish in a vertical position.

Aspect 26: A method comprising:
positioning cellular media within the insert of the apparatus of any one of aspects 20-25; and
applying an electric field to the cellular media.

Aspect 27: The method of aspect 26, further comprising:
directing condensate radially outwardly from the inner portion of the cover to the outer portion of the cover.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A cover having a central axis and a circumference, the cover being configured to overlie an interior space defined by a container, the cover comprising:
a base having a center point that is intersected by the central axis of the cover, wherein the base comprises:
an outer portion extending radially inwardly from the circumference toward the central axis, wherein the outer portion defines a bottom surface of the cover; and
an inner portion positioned radially inwardly of the outer portion, wherein the inner portion comprises an apex that is upwardly spaced from the outer portion along the central axis,
wherein the inner portion of the base is configured to direct condensate radially outwardly from the apex toward the outer portion of the base.

2. The cover of claim 1, wherein the inner portion of the base extends radially inwardly from and is circumferentially surrounded by the outer portion of the base.

3. The cover of claim 1, wherein the apex of the base is intersected by the central axis of the cover.

4. The cover of claim 3, wherein the inner portion of the base has a conical shape.

5. The cover of claim 3, wherein the inner portion of the base has one or more sloped surfaces that extend upwardly from the outer portion of the base.

6. The cover of claim 5, wherein within respective vertical planes that intersect the apex, opposing sloped surfaces of the inner portion of the base form an obtuse angle.

7. The cover of claim 6, wherein the obtuse angle ranges from about 125 degrees to about 175 degrees.

8. The cover of claim 1, further comprising a peripheral wall extending from the outer portion of the base and defining the circumference of the cover.

9. The cover of claim 8, wherein the peripheral wall extends upwardly from the outer portion of the base.

10. The cover of claim 1, further comprising a plurality of projections that extends downwardly from the outer portion of the base, wherein the plurality of projections are equally radially spaced from the central axis.

11. The cover of claim 8, wherein the peripheral wall extends downwardly from the outer portion of the base.

12. The cover of claim 1, wherein the cover does not comprise a peripheral wall that extends upwardly or downwardly from the outer portion of the base, and wherein the cover comprises at least one projection that extends downwardly from the outer portion of the base.

13. An apparatus comprising:
a dish having a peripheral wall that defines a top surface of the dish; and
a cover having a central axis and a circumference, the cover comprising:
a base having a center point that is intersected by the central axis of the cover, wherein the base comprises:
an outer portion extending radially inwardly from the circumference toward the central axis, wherein the outer portion defines a bottom surface of the cover; and
an inner portion positioned radially inwardly of the outer portion, wherein the inner portion comprises an apex that is upwardly spaced from the outer portion along the central axis,
wherein the inner portion of the base is configured to direct condensate radially outwardly from the apex toward the outer portion of the base,
wherein the bottom surface of the cover rests on the top surface of the dish.

14. The apparatus of claim 13, further comprising an insert positioned within the dish, the insert being configured to receive cellular media, wherein the apex of the cover overlies the insert.

15. The apparatus of claim 14, wherein the outer portion of the base of the cover does not overlie the insert.

16. The apparatus of claim 13, wherein the cover comprises at least one projection that extends downwardly from the outer portion of the base of the cover, and wherein each projection of the at least one projection has an inwardly facing surface that is positioned radially outwardly of the peripheral wall of the dish.

17. The apparatus of claim 13, wherein the cover comprises a peripheral wall that extends downwardly from the outer portion of the base of the cover, and wherein the peripheral wall of the cover is positioned radially outwardly of the peripheral wall of the dish.

18. The apparatus of claim 13, further comprising a holder that supports the dish in a vertical position.

19. A method comprising:
positioning cellular media within an insert of an apparatus comprising:
a dish having a peripheral wall that defines a top surface of the dish; and
a cover having a central axis and a circumference, the cover comprising:
a base having a center point that is intersected by the central axis of the cover, wherein the base comprises:
an outer portion extending radially inwardly from the circumference toward the central axis, wherein the outer portion defines a bottom surface of the cover; and
an inner portion positioned radially inwardly of the outer portion, wherein the inner portion comprises an apex that is upwardly spaced from the outer portion along the central axis,
wherein the inner portion of the base is configured to direct condensate radially outwardly from the apex toward the outer portion of the base,
wherein the bottom surface of the cover rests on the top surface of the dish; and
an insert positioned within the dish, the insert being configured to receive cellular media, wherein the apex of the cover overlies the insert; and
applying an electric field to the cellular media.

20. The method of claim 19, further comprising:
directing condensate radially outwardly from the inner portion of the cover to the outer portion of the cover.
